(19) United States
(12) Patent Application Publication
(10) Pub. No.: US 2009/0048303 A1
(43) Pub. Date: Feb. 19, 2009

(54) Indole-2-carboxamidine derivatives as nmda receptor antago

(76) Inventors: Istvan Borza, Budapest (HU); Csilla Horvath, Budapest (HU); Sandor Farkas, Budapest (HU); Jozsef Nagy, Budapest (HU); Sandor Kolok, Budapest (HU)

Correspondence Address: K.F. ROSS P.C., 5683 RIVERDALE AVENUE, SUITE 203 BOX 900, BRONX, NY, 10471-0900, US

(21) Appl. No.: 11/658,787

(22) PCT Filed: Jul. 21, 2005
(86) PCT No.: PCT/HU2005/000078

§ 371 Date: Jan. 25, 2007

(30) Foreign Application Priority Data
Jul. 29, 2004 (HU) P0401523

(51) Int. Cl. A61K 31/4439; C07D 209/14; A61K 31/404; C07D 401/02

(52) U.S. Cl. 514/339; 548/505; 514/415; 546/277.4

(57) ABSTRACT

The present invention relates therefore first to new indole-2-carboxamidine derivatives of formula (I)—wherein the meaning of X is hydrogen or halogen atom, C₁-C₄ alkyl, C₁-C₄ alkoxy, trifluoromethyl group, Y, V and Z independently are hydrogen or halogen atom, hydroxy, cyano, C₁-C₄ alkylsulfonamido optionally substituted by a halogen atom or halogen atoms, C₁-C₄ alkanoylamido optionally substituted by a halogen atom or halogen atoms, trifluoromethyl, trifluoromethoxy, C₁-C₄ alkyl, C₁-C₄ alkoxy group, or the neighboring V and Z groups in given case together with one or more identical or different additional hetero atom and —CH═ and/or —CH₂— groups can form an optionally substituted 4-7 membered homo- or heterocyclic ring, preferably benzene, dioxolane ring, A, B and C independently are substituted carbon atom or one of them is nitrogen atom, and the salts thereof. Further objects of the invention are the processes for producing indole-2-carboxamidine derivatives of formula (I), and the pharmaceutical manufacture of medicaments containing these compounds, as well as the process of treatments with these compounds, which means administering to a mammal to be treated—including human—effective amount/amounts of indole-2-carboxamidine derivatives of formula (I) of the present invention as such or as medicament. The new indole-2-carboxamidine derivatives of formula (I) of the present invention are highly effective and selective antagonists of NMDA receptor, and moreover most of the compounds are selective antagonist of NR2B subtype of NMDA receptor.

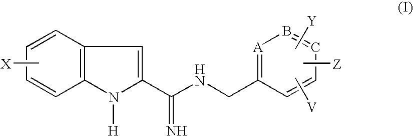

The invention relates to new indole-2-carboxamidine derivatives which are antagonists of NMDA receptor or are intermediates for preparing thereof.

### BACKGROUND OF THE INVENTION

N-methyl-D-aspartate (NMDA) receptors are ligand-gated cation-channels widely expressed in the central nervous system. NMDA receptors are involved in developmental and plastic changes of the central nervous system. Overactivation of NMDA receptors by glutamate, their natural ligand, can lead to calcium overload of cells. This triggers a cascade of intracellular events that alters the cell function and ultimately may lead to death of neurones [TINS, 10, 299-302 (1987)]. Antagonists of the NMDA receptors may be used for treating many disorders that are accompanied with excess release of glutamate.

The NMDA receptors are heteromeric assemblies built up from at least one NR1 subunit together with one or more of the four NR2 subunits (NR2A-D). Recently, NR3A and NR3B have been reported. Both spatial distributions in the CNS and the pharmacological sensitivity of NMDA receptors built up from various NR2 subunits are different. Particularly interesting of these is the NR2B subunit due to its restricted distribution (highest densities in the forebrain and substantia gelatinosa of the spinal cord). Compounds selective for this subtype are available and have been proved to be effective in animal models of stroke [Stroke, 28, 2244-2251 (1997)], traumatic brain injury [Brain Res., 792, 291-298 (1998)], Parkinson's disease [Exp. Neurol., 163, 239-243 (2000)], neuropathic and inflammatory pain [Neuropharmacology, 38, 611-623 (1999)]. Moreover, NR2B subtype selective antagonists of NMDA receptors are expected to possess more favorable side effect profile than non-selective antagonists of NMDA receptors. The NR2B selective compound CP-101,606 when examined in a clinical trial was well tolerated and did not have the undesirable effects characteristic of non-selective antagonists, namely psychotomimetic effects, hallucinations, dysphoria at a dose that reduced neuropathic pain (C. N. Sang, et al. Program No. 814.9. 2003 Abstract Viewer/Itinerary Planner. Washington, D.C.: Society for Neuroscience, 2003. Online).

NR2B subtype selective NMDA antagonism can be achieved with compounds that specifically bind to, and act on, an allosteric modulatory site of the NR2B subunit containing receptors. This binding site can be characterised by displacement (binding) studies with specific radioligands, such as [³H]-Ro 25,6981 [J. Neurochem., 70, 2147-2155 (1998)].

Close structure analogs of the carboxylic acid amidine derivatives of formula (I) are unknown from the literature. Although, some different carboxamidine derivatives are described as NR2B subtype selective NMDA antagonists in the following publications:

Cinnamamidines are described in patent No. WO 200198262 and Bioorg. Med. Chem. Letters, 13, 693-696. (2003).

Benzamidines are described in patent No. WO 200067751 and Bioorg. Med. Chem. Letters, 13, 693-696. (2003) as well as Bioorg. Med. Chem. Letters, 13, 697-700. (2003).

### SUMMARY OF THE INVENTION

Surprisingly it was found that the new indole-2-carboxamidine derivatives of formula (I) of the present invention are functional antagonists of NMDA receptors of rat cortical neurons with IC50 below 1 micromole. This effect is due to their inhibition of NR2B containing receptors, since they are ineffective on NR2A subunit containing NMDA receptors at 10 micromole. Therefore, they are believed to be NR2B subtype specific NMDA antagonists, and possess therapeutic utility.

### DETAILED DESCRIPTION OF THE INVENTION

The present invention relates therefore first to new indole-2-carboxamidine derivatives of formula (I)

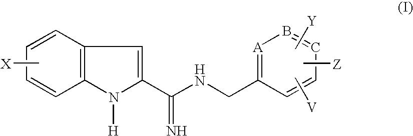

wherein the meaning of
X is hydrogen or halogen atom, C₁-C₄ alkyl, C₁-C₄ alkoxy, trifluoromethyl group,
Y, V and Z independently are hydrogen or halogen atom, hydroxy, cyano, C₁-C₄ alkylsulfonamido optionally substituted by a halogen atom or halogen atoms, C₁-C₄ alkanoylamido optionally substituted by a halogen atom or halogen atoms, trifluoromethyl, trifluoromethoxy, C₁-C₄ alkyl, C₁-C₄ alkoxy group, or
the neighboring V and Z groups in given case together with one or more identical or different additional hetero atom and —CH═ and/or —CH₂— groups can form an optionally substituted 4-7 membered homo- or heterocyclic ring, preferably benzene, dioxolane ring,
   A, B and C independently are substituted carbon atom or one of them is nitrogen atom, and the salts thereof.

Further objects of the invention are the processes for producing indole-2-carboxamidine derivatives of formula (I), and the pharmaceutical manufacture of medicaments containing these compounds, as well as the process of treatments with these compounds, which means administering to a mammal to be treated—including human—effective amount/amounts of indole-2-carboxamidine derivatives of formula (I) of the present invention as such or as medicament.

The new indole-2-carboxamidine derivatives of formula (I) of the present invention are highly effective and selective antagonists of NMDA receptor, and moreover most of the compounds are selective antagonist of NR2B subtype of NMDA receptor.

According to the invention the indole-2-carboxamidine derivatives of formula (I) are synthesized by reacting a carboximidic acid alkyl ester salt of formula (II)

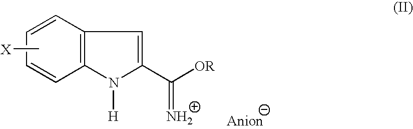

wherein the meaning of X is as given in claim 1, Anion means one equivalent of an anion, R is C₁-C₄ alkyl—with an aralkylamine of formula (III)

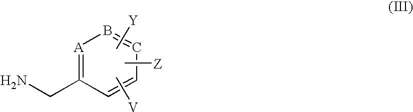

wherein the meaning of A, B, C, Y, V, Z are as given before for the formula (I),

then the so obtained indole-2-carboxamidine derivatives of formula (I)—wherein the meaning of A, B, C, Y, V, Z are as defined for the formula (I)—in given case are transformed into another compounds of formula (I) by introducing new substituents and/or modifying or removing the existing ones, and/or by forming salt and/or by liberating the compound from salts by known methods.

The compounds of this invention are readily prepared by reacting the appropriate carboximidic acid alkyl ester hydrochloride with an appropriate aralkylamine in a reaction-inert solvent at a suitable temperature, in the presence of a base. Representative solvents for these reactions are ethanol, methanol, methylene chloride, ethylene dichloride, tetrahydrofuran.

The desired carboximidic acid alkyl ester hydrochlorides (e.g. ethyl ester) are conveniently prepared by Pinner reaction i.e. by reacting the corresponding nitril with saturated hydrochloric acid solution of alcohol. The nitril reactants needed for the preparation of the carboximidic acid alkyl ester hydrochloride reactants are prepared by reaction of the corresponding amides with phosphorus oxychloride. The acid amides are prepared by amidation of the corresponding acid chlorides according to well known procedures. The acid chlorides are prepared by reaction of the appropriate carboxylic acid with thionyl chloride, the latter generally serving as reactant and solvent as well. In the reaction of compound of formula (II) with compound of formula (III) the applied base is the molar excess of the reagent aralkylamine of formula (III). The necessary reaction time is 1-24 h, preferably 6 h. The preferred reaction temperature is from about 20° C. to about 30° C. The reaction mixture is purified by column chromatography using Kieselgel 60 (Merck) as adsorbent and a proper eluent. The proper fractions are acidified with hydrochloric acid and concentrated to give the hydrochloric acid salt of the pure product. The quality and the quantity of the product are determined by HPLC-MS method.

The 1H-indole-2-carboxylic acids needed for the preparation of the carboximidic acid alkyl ester hydrochloride reactants of formula (II) and the benzil amines of formula (III) are either commercially available.

### Experimental Protocols

### Expression of Recombinant NMDA Receptors

To prove NR2B selectivity of our compounds, we tested them on cell lines stably expressing recombinant NMDA receptors with subunit compositions of NR1(−3)/NR2A. cDNAs of human NR1(−3) and NR2A subunits subcloned into inducible mammalian expression vectors were introduced into HEK 293 cells lacking NMDA receptors using a cationic lipid-mediated transfection method [Biotechniques, 1997 May; 22(5): 982-7; Neurochemistry International, 43, 19-29. (2003)]. Resistance to neomycin and hygromycin was used to screen for clones possessing both vectors and monoclonal cell lines were established from the clones producing the highest response to NMDA exposure. Compounds were tested for their inhibitory action on NMDA evoked cytosolic calcium elevations in fluorescent calcium measurements. Studies were performed 48-72 h after addition of the inducing agent. Ketamine (500 μM) was also present during the induction in order to prevent cytotoxicity.

Assessment of NMDA Antagonist Potency In Vitro by Measurement of Intracellular Calcium Concentration with a Plate Reader Fluorimeter in Rat Cortical Cell Culture

The intracellular calcium measurements were carried out on primary neocortical cell cultures derived from 17 day old Charles River rat embryos (for the details on the preparation of neocortical cell culture see Johnson, M. I.; Bunge, R. P. (1992): Primary cell cultures of peripheral and central neurons and glia. In: Protocols for Neural Cell Culture, eds: Fedoroff, S.; Richardson A., The Humana Press Inc., 51-75.) After isolation the cells were plated onto standard 96-well microplates and the cultures were maintained in an atmosphere of 95% air-5% CO₂ at 37° C. until the calcium measurements.

The cultures were used for the intracellular calcium measurements after 3-7 days in vitro. Before the measurement the cells were loaded with a fluorescent Ca²⁺-sensitive dye, Fluo-4/AM (2 μM). To stop the loading the cells were washed twice with the solution used for the measurement (140 mM NaCl, 5 mM KCl, 2 mM CaCl₂, 5 mM HEPES, 5 mM HEPES-Na, 20 mM glucose, 10 μM glycine, pH=7.4). After washing the test compounds were added to the cells in the above solution (90 μl/well). Intracellular calcium measurements were carried out with a plate reader fluorimeter: elevation of Fluo-4-fluorescence and so, intracellular calcium was induced by application of 40 μM NMDA. Inhibitory potency of the test compounds was assessed by measuring the reduction in the calcium elevation in the presence of different concentrations of the compounds.

Dose-response curves and IC₅₀ values were calculated by using data derived from at least three independent experiments. Inhibitory potency of a compound at a single concentration point was expressed as percent inhibition of the NMDA response. Sigmoidal concentration-inhibition curves were fit to the data and IC₅₀ values were determined as the concentration that produces half of the maximal inhibition caused by the compound.

In Table 1, NR2B antagonist potencies of the most effective compounds of this invention determined in this test are listed. The results with the selective NR2B antagonist CI-1041 and the non-selective NMDA receptor antagonist MK-801, used as reference compounds, are given in Table 2.

TABLE 1
NMDA antagonist activity of compounds measured by fluorimetric
method on cortical cells or NR1-3/NR2A subunit expressing cells

| Compound of | Cortical cells | | NR1-3/NR2A | |
|---|---|---|---|---|
| Table 3 | IC₅₀ [nM] | N | % inhibition at 15 μM | n |
| 36 | 5.4 | 3 | 32.1 | 1 |
| 147 | 26 | 5 | 21.7 | 1 |
| 134 | 100 | 4 | 6.1 | 1 |
| 19 | 75 | 5 | | |
| 57 | 211 | 3 | | |
| 59 | 51 | 3 | | |
| 81 | 432 | 4 | | |
| 83 | 369 | 4 | | |
| 87 | 422 | 6 | | |
| 92 | 319 | 4 | | |
| 94 | 213 | 4 | | |
| 97 | 125 | 4 | | |
| 118 | 224 | 5 | | |
| 119 | 401 | 3 | | |
| 123 | 129 | 4 | | |
| 127 | 373 | 6 | | |
| 128 | 220 | 3 | | |
| 129 | 52 | 4 | | |
| 130 | 266 | 5 | | |
| 131 | 57 | 4 | | |
| 25 | 277 | 5 | | |
| 26 | 418 | 6 | | |
| 29 | 232 | 3 | | |
| 30 | 224 | 3 | | |
| 31 | 143 | 4 | | |
| 32 | 170 | 4 | | |
| 35 | 341 | 3 | | |
| 67 | 359 | 3 | | |
| 68 | 160 | 4 | | |
| 70 | 594 | 5 | | |
| 71 | 453 | 3 | | |
| 72 | 17 | 3 | | |
| 103 | 257 | 4 | | |
| 107 | 493 | 3 | | |
| 126 | 519 | 5 | | |
| 109 | 218 | 3 | | |
| 110 | 702 | 3 | | |
| 111 | 417 | 3 | | |
| 112 | 69 | 4 | | |
| 138 | 232 | 3 | | |
| 139 | 397 | 4 | | |
| 142 | 202 | 3 | | |
| 143 | 158 | 3 | | |
| 145 | 318 | 3 | | |
| 146 | 287 | 4 | | |
| 108 | 742 | 4 | | |
| 106 | 198 | 3 | | |
| 38 | 159 | 3 | | |
| 73 | 209 | 5 | | |

TABLE 2
NMDA antagonist activity of reference compounds
measured by fluorimetric method on cortical cells
or NR1-3/NR2A subunit expressing cells

| Code of | Cortical cells | | NR1-3/NR2A | |
|---|---|---|---|---|
| reference compounds | IC₅₀ [nM] | n | % inhibition at 10 μM | n |
| CI-1041 | 6.6 | 4 | 21.0 | 1 |
| Co-101244 | 23 | 3 | −8.7 | 1 |
| EMD 95885 | 35 | 1 | 0.1 | 1 |
| CP 101,606 | 41 | 3 | 2.5 | 1 |
| Ro 25.6981 | 159 | 4 | 1.0 | 1 |
| Erythro-ifenprodil | 483 | 5 | −2.7 | 1 |
| MK-801 | 37 | 3 | IC₅₀ = 386 nM | 2 |

The reference compounds are as follows:
CI-1041: 6-{2-[4-(4-fluoro-benzyl)-piperidin-1-yl]-ethanesulfinyl}-3H-benzooxazol-2-one
Co 101244: 1-[2-(4-hydroxyphenoxy)ethyl]-4-hydroxy-4-(4-methylbenzyl)piperidine
EMD 95885: 6-[3-(4-fluorobenzyl)piperidine-1-yl]propionyl]-2,3-dihydro-benzoxazol-2-on
CP-101,606: (1S,2S)-1-(4-hydroxyphenyl)-2-(4-hydroxy-4-phenylpiperidine-1-yl)-1-propanol
Co-111103: 1-[2-(4-hydroxyphenoxy)ethyl]-4-(4-fluorobenzyl)piperidine
Ro 256981: R-(R*,S*)-1-(4-hydroxyphenyl)-2-methyl-3-[4-(phenylmethyl)piperidin-1-yl]-1-propanol.
Ifenprodil: erythro-2-(4-benzylpiperidino)-1-(4-hydroxyphenyl)-1-propanol
MK-801: (+)-5-Methyl-10,11-dihydro-5H-dibenzo[a,d]cyclohepten-5,10-imine

Disorders which may be beneficially treated with NMDA antagonists acting at NR2B site, as reviewed recently by Loftis [Pharmacology & Therapeutics, 97, 55-85 (2003)] include schizophrenia, Parkinson's disease, Huntington's disease, excitotoxicity evoked by hypoxia and ischemia, seizure disorders, drug abuse, and pain, especially neuropathic, inflammatory and visceral pain of any origin [Eur. J. Pharmacol., 429, 71-78 (2001)].

Due to their reduced side effect liability compared to non-selective NMDA antagonists, NR2B selective antagonists may have utility in diseases where NMDA antagonist may be effective, such as amyotrophic lateral sclerosis [Neurol. Res., 21, 309-12 (1999)], withdrawal syndromes of e.g. alcohol, opioids or cocaine [Drug and Alcohol Depend., 59′ 1-15 (2000)], muscular spasm [Neurosci. Lett., 73, 143-148 (1987)], dementia of various origins [Expert Opin. Investig. Drugs, 9, 1397-406 (2000)], anxiety, depression, migraine, hypoglycemia, degenerative disorders of the retina (e.g. CMV retinitis), glaucoma, asthma, tinnitus, hearing loss [Drug News Perspect 11, 523-569 (1998) and WO 00/00197 international patent application].

Accordingly, effective amounts of the compounds of the invention may be beneficially used for the treatment of traumatic injury of brain or spinal cord, tolerance and/or dependence to opioid treatment of pain, development of tolerance, decrease of abuse potential and withdrawal syndromes of drugs of abuse e.g. alcohol, opioids or cocaine, ischernic CNS disorders, chronic neurodegenerative disorders, such as e.g. Alzheimer's disease, Parkinson's disease, Huntington's disease, pain and chronic pain states, such as e.g. neuropathic pain.

The compounds of the invention as well as their pharmaceutically acceptable salts can be used as such or suitably in the form of pharmaceutical compositions. These compositions (drugs) can be in solid, liquid or semiliquid form and pharmaceutical adjuvant and auxiliary materials can be added, which are commonly used in practice, such as carriers, excipients, diluents, stabilizers, wetting or emulsifying agents, pH- and osmotic pressure-influencing, flavoring or aromatizing, as well as formulation-promoting or formulation-providing additives.

The dosage required to exert the therapeutical effect can vary within wide limits and will be fitted to the individual requirements in each of the particular cases, depending on the stage of the disease, the condition and the bodyweight of the patient to be treated, as well as the sensitivity of the patient against the active ingredient, route of administration and number of daily treatments. The actual dose of the active ingredient to be used can safely be determined by the attending physician skilled in the art in the knowledge of the patient to be treated.

The pharmaceutical compositions containing the active ingredient according to the present invention usually contain 0.01 to 100 mg of active ingredient in a single dosage unit. It is, of course possible that the amount of the active ingredient in some compositions exceeds the upper or lower limits defined above.

The solid forms of the pharmaceutical compositions can be for example tablets, dragées, capsules, pills or lyophilized powder ampoules useful for the preparation of injections. Liquid compositions are the injectable and infusable compositions, fluid medicines, packing fluids and drops. Semiliquid compositions can be ointments, balsams, creams, shaking mixtures and suppositories.

For the sake of a simple administration it is suitable if the pharmaceutical compositions comprise dosage units containing the amount of the active ingredient to be administered once, or a few multiples or a half, third or fourth part thereof. Such dosage units are e.g. tablets, which can be powdered with grooves promoting the halving or quartering of the tablet in order to exactly administer the required amount of the active ingredient.

Tablets can be coated with an acid-soluble layer in order to assure the release of the active ingredient content after leaving the stomach. Such tablets are enteric-coated. A similar effect can be achieved also by encapsulating the active ingredient.

The pharmaceutical compositions for oral administration can contain e.g. lactose or starch as excipients, sodium carboxymethylcellulose, methylcellulose, polyvinyl pyrrolidone or starch paste as binders or granulating agents. Potato starch or microcrystalline cellulose is added as disintegration agents, but ultraamylopectin or formaldehyde casein can also be used. Talcum, colloidic silicic acid, stearin, calcium or magnesium stearate can be used as antiadhesive and lubricants.

The tablet can be manufactured for example by wet granulation, followed by pressing. The mixed active ingredients and excipients, as well as in given case part of the disintegrants are granulated with an aqueous, alcoholic or aqueous alcoholic solution of the binders in an appropriate equipment, then the granulate is dried. The other disintegrants, lubricants and antiadhesive agents are added to the dried granulate, and the mixture is pressed to a tablet. In given case the tablets are made with halving groove to ease the administration.

The tablets can be made directly from the mixture of the active ingredient and the proper auxiliaries by pressing. In given case, the tablets can be coated by using additives commonly used in the pharmaceutical practice, for example stabilizers, flavoring, coloring agents, such as sugar, cellulose derivatives (methyl- or ethylcellulose, sodium carboxymethylcellulose, etc), polyvinyl pyrrolidone, calcium phosphate, calcium carbonate, food coloring agents, food laces, aroma agents, iron oxide pigments, etc. In the case of capsules the mixture of the active ingredient and the auxiliaries is filled into capsules.

Liquid oral compositions, for example suspensions, syrups, elixirs can be made by using water, glycols, oils, alcohols, coloring and flavoring agents.

For rectal administration the composition is formulated in suppositories or clysters. The suppository can contain beside the active ingredient a carrier, so called adeps pro suppository. Carriers can be vegetable oils, such as hydrogenated vegetable oils, triglycerides of C₁₂-C₁₈ fatty acids (preferably the carriers under the trade name Witepsol). The active ingredient is homogeneously mixed with the melted adeps pro suppository and the suppositories are moulded.

For parenteral administration the composition is formulated as injection solution. For manufacturing the injection solution the active ingredients are dissolved in distilled water and/or in different organic solvents, such as glycolethers, in given case in the presence of solubilizers, for example polioxyethylensorbitane-monolaurate, -monooleate, or monostearate (Tween 20, Tween 60, Tween 80). The injection solution can also contain different auxiliaries, such as conserving agents, for example ethylendiamine tetraacetate, as well as pH adjusting agents and buffers and in given case local anaesthetic, e.g. lidocain. The injection solution containing the active ingredient of the invention is filtered before it is filled into ampoules, and it is sterilized after filling.

If the active ingredient is hygroscopic, then it can be stabilized by liophylization.

The following examples illustrate the invention without the intention of limitation anyway.

### Characterization Method

Compounds of the present invention were characterized by high performance liquid chromatography coupled to mass selective detector (LC/MS) using BP 1100 Binary Gradient chromatography system with Microplate Sampler (Agilent, Waldbronn), controlled by ChemStation software. HP diode array detector was used to acquire uV spectra at 225 and 240 nm. All experiments were performed using HP MSD (Agilent, Waldbronn) single quadruple spectrometer equipped with an electrospray ionization source to determine the structure.

The synthesized products were dissolved in 1 ml of DMSO (Aldrich, Germany). 100 μl of each solution was diluted with DMSO to 1000 μl volume. Analytical chromatographic experiments were performed on Discovery RP C-16 Amide, 5 cm×4.6 mm×5 μm column from Supelco (Bellefonte, Pa.) with a flow rate of 1 ml/minute for qualification. The obtained compounds were characterized by their k′ value (purity, capacity factor). k′ factors are evaluated by the following formula:

k′=(t_{R}−t₀)/t₀

where k′=capacity factor, t_{R}=retention time and t₀=eluent retention time.

The A eluent was trifluoroacetic acid (TFA) (Sigma, Germany) containing 0.1% water, the B eluent was 95% acetonitrile (Merck, Germany) containing 0.1% TFA and 5% A eluent. Gradient elution was used, starting with 100% A eluent and processing to 100% B eluent over a period of 5 minutes.

The process according to our invention is illustrated in detail by the following not limiting examples.

### EXAMPLE 1

### N-Benzyl-1H-indole-2-carboxamidine hydrochloride

### 1a) 1H-Indole-2-carboxylic acid amide

A mixture of 10.0 g (62 mmol) of 1H-indole-2-carboxylic acid (Aldrich), 10.0 ml (76 mmol) of thionyl chloride, 100 ml of chloroform and 1 drop of dimethylformaimide is refluxed for 2 h. The reaction mixture is cooled to 20° C., poured into a mixture of 100 ml of 25% ammonia solution and 100 g of ice, then stirred for 2 h. The precipitated product is filtered off and washed with water to yield 8.96 g (90%) of the title compound. Mp.: 231-232° C.

### 1b) 1H-Indole-2-carbonitrile

A mixture of 8.96 g (56 mmol) of 1H-indole-2-carboxylic acid amide, 26.0 ml (279 mmol) of phosphorus oxychloride and 230 ml of chloroform is refluxed for 2 h. The reaction mixture is cooled to 20° C., poured into 100 ml of water and stirred for 1 h. After separation the organic layer is dried over sodium sulfate and concentrated. The residue is purified by column chromatography using Kieselgel 60 (Merck) as adsorbent and hexane-ethyl acetate=4:1 as eluent to yield 5.28 g (66.4%) of the title compound. Mp.: 94-95° C.

### 1c) 1H-indole-2-carboximidic acid ethyl ester hydrochloride

To a solution of 20 ml of saturated hydrochloric acid in ethanol 2.23 g (15.7 mmol) of 1H-indole-2-carbonitrile is added. The reaction mixture is stirred at 20° C. for 2 h, then concentrated and the residue is crystallized with ether to yield 3.1 g (82%) of the title compound. Mp.: 170° C.

### 1d) N-Benzyl-1H-indole-2-carboxamidine hydrochloride

To a solution of 32.1 mg (0.3 mmol) of benzylamine in 1 ml of ethanol, 28.2 mg (0.15 mmol) of 1H-indole-2-carboximidic acid ethyl ester hydrochloride in 1 ml of ethanol is added. The reaction mixture is stirred at room temperature for 6 h, and then concentrated. The residue is purified by column chromatography using Kieselgel 60 (Merck) as adsorbent and toluene:methanol=4:1 as eluent. The fractions containing the product are acidified with hydrochloric acid and concentrated. The quality and the quantity of the product are determined by HPLC-MS method as described above. k′=2.46.

The substituted 1H-indole-2-carboximidic acid ethyl ester hydrochloride derivatives covered in the Table 3, which are representatives of the compounds of formula (I) are prepared from the corresponding 1H-indole-2-carboxylic acid according to the method described in Example 1 and are characterized using the above characterizing method.

TABLE 3

| | |
|---|---|
| | (I) |
| 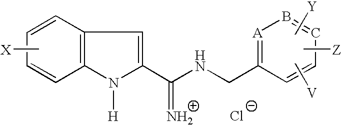 | |

| No. | 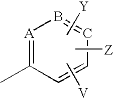 | X | k' |
|---|---|---|---|
| 1 | 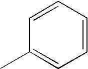 | H | 2.46 |
| 2 | 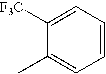 | H | 2.78 |
| 3 | 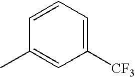 | H | 3.01 |
| 4 | 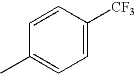 | H | 3.02 |
| 5 | 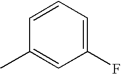 | H | 2.51 |
| 6 | 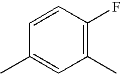 | H | 2.56 |
| 7 | 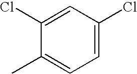 | H | 3.01 |
| 8 | 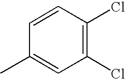 | H | 3.14 |
| 9 | 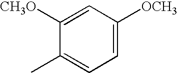 | H | 2.82 |
| 10 | 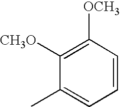 | H | 2.66 |
| 11 | 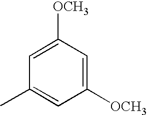 | H | 2.69 |
| 12 | 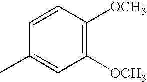 | H | 2.47 |
| 13 | 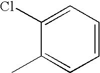 | H | 2.67 |
| 14 | 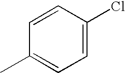 | H | 2.76 |
| 15 | 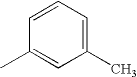 | H | 2.73 |
| 16 | 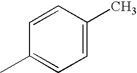 | H | 2.8 |
| 17 | 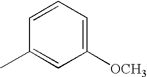 | H | 2.61 |
| 18 | 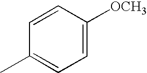 | H | 2.58 |
| 19 | 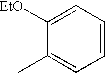 | H | 2.85 |
| 20 | 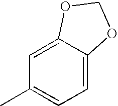 | H | 2.56 |
| 21 | 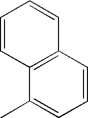 | H | 4.01 |
| 22 | 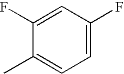 | H | 3.57 |
| 23 | 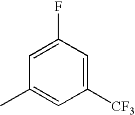 | H | 3.98 |
| 24 | 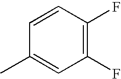 | H | 3.61 |
| 25 | 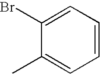 | H | 3.86 |
| 26 | 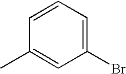 | H | 3.83 |
| 27 | 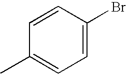 | H | 3.88 |
| 28 | 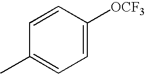 | H | 3.99 |
| 29 | 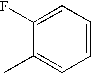 | H | 3.54 |
| 30 | 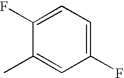 | H | 3.53 |
| 31 | 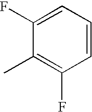 | H | 3.52 |
| 32 | 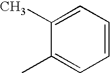 | H | 3.73 |
| 33 | 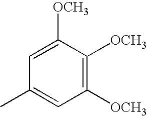 | H | 3.35 |
| 34 | 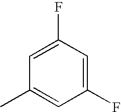 | H | 3.64 |
| 35 | 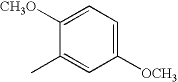 | H | 3.66 |
| 36 | 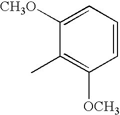 | H | 3.67 |
| 37 | 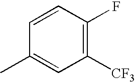 | H | 3.671 |
| 38 | 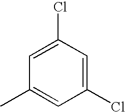 | H | 3.913 |
| 39 | 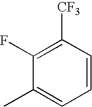 | H | 3.94 |
| 40 | 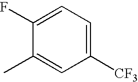 | H | 3.67 |
| 41 | 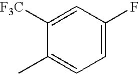 | H | 3.45 |
| 42 | 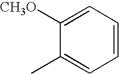 | H | 5.23 |
| 43 | 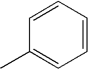 | 5-F | 2.53 |
| 44 | 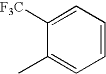 | 5-F | 2.91 |
| 45 | 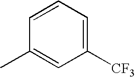 | 5-F | 3.09 |
| 46 | 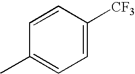 | 5-F | 3.06 |
| 47 | 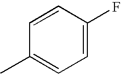 | 5-F | 2.64 |
| 48 | 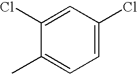 | 5-F | 3.13 |
| 49 | 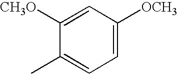 | 5-F | 2.91 |
| 50 | 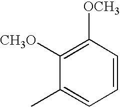 | 5-F | 2.77 |
| 51 | 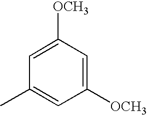 | 5-F | 2.81 |
| 52 | 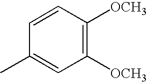 | 5-F | 2.61 |
| 53 | 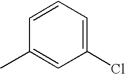 | 5-F | 2.91 |
| 54 | 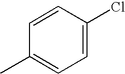 | 5-F | 2.77 |
| 55 | 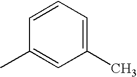 | 5-F | 2.81 |
| 56 | 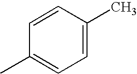 | 5-F | 2.61 |
| 57 | 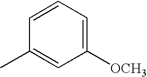 | 5-F | 2.91 |
| 58 | 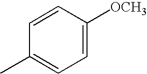 | 5-F | 2.77 |
| 59 | 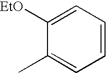 | 5-F | 2.81 |
| 60 | 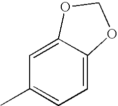 | 5-F | 2.61 |
| 61 | 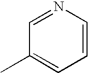 | 5-F | 0.75 |
| 62 | 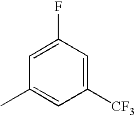 | 5-F | 3.98 |
| 63 | 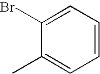 | 5-F | 3.87 |
| 64 | 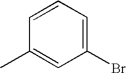 | 5-F | 3.9 |
| 65 | 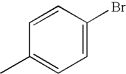 | 5-F | 3.83 |
| 66 | 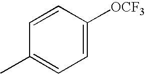 | 5-F | 4.05 |
| 67 | 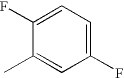 | 5-F | 3.66 |
| 68 | 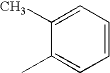 | 5-F | 3.7 |
| 69 | 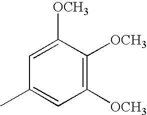 | 5-F | 3.16 |
| 70 | 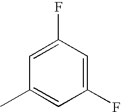 | 5-F | 3.75 |
| 71 | 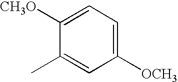 | 5-F | 3.7 |
| 72 | 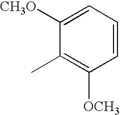 | 5-F | 3.8 |
| 73 | 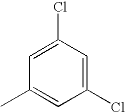 | 5-F | 3.947 |
| 74 | 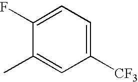 | 5-F | 3.768 |
| 75 | 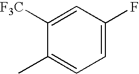 | 5-F | 3.689 |
| 76 | 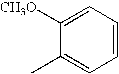 | 5-F | 5.63 |
| 77 | 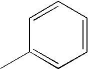 | 5-Cl | 2.79 |
| 78 | 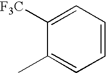 | 5-Cl | 3.08 |
| 79 | 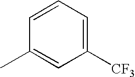 | 5-Cl | 3.29 |
| 80 | 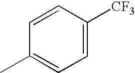 | 5-Cl | 3.26 |
| 81 | 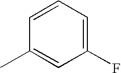 | 5-Cl | 2.91 |
| 82 | 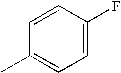 | 5-Cl | 2.91 |
| 83 | 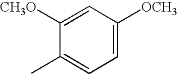 | 5-Cl | 3.02 |
| 84 | 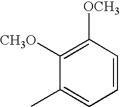 | 5-Cl | 2.56 |
| 85 | 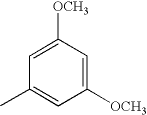 | 5-Cl | 3.07 |
| 86 | 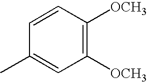 | 5-Cl | 2.86 |
| 87 | 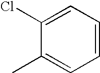 | 5-Cl | 3.03 |
| 88 | 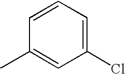 | 5-Cl | 3.1 |
| 89 | 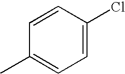 | 5-Cl | 3.12 |
| 90 | 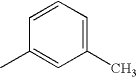 | 5-Cl | 3.03 |
| 91 | 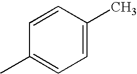 | 5-Cl | 3.03 |
| 92 | 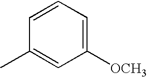 | 5-Cl | 2.94 |
| 93 | 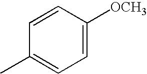 | 5-Cl | 2.95 |
| 94 | 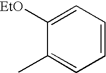 | 5-Cl | 3.17 |
| 95 | 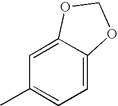 | 5-Cl | 2.9 |
| 96 | 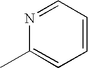 | 5-Cl | 1.94 |
| 97 | 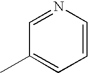 | 5-Cl | 1.39 |
| 98 | 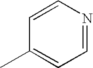 | 5-Cl | 1.23 |
| 99 | 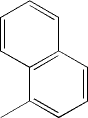 | 5-Cl | 4.23 |
| 100 | 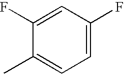 | 5-Cl | 3.88 |
| 101 | 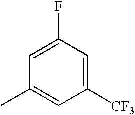 | 5-Cl | 4.055 |
| 102 | 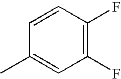 | 5-Cl | 3.91 |
| 103 | 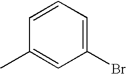 | 5-Cl | 4.07 |
| 104 | 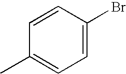 | 5-Cl | 4.01 |
| 105 | 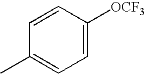 | 5-Cl | 4.23 |
| 106 | 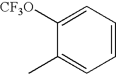 | 5-Cl | 3.762 |
| 107 | 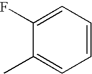 | 5-Cl | 3.86 |
| 108 | 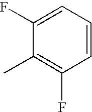 | 5-Cl | 3.66 |
| 109 | 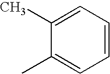 | 5-Cl | 3.89 |
| 110 | 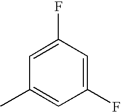 | 5-Cl | 3.86 |
| 111 | 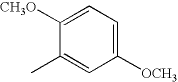 | 5-Cl | 3.85 |
| 112 | 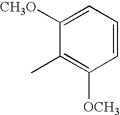 | 5-Cl | 3.9 |
| 113 | 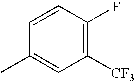 | 5-Cl | 4.13 |
| 114 | 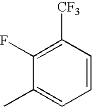 | 5-Cl | 4.04 |
| 115 | 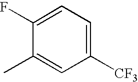 | 5-Cl | 4.08 |
| 116 | 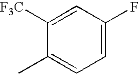 | 5-Cl | 3.97 |
| 117 | 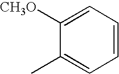 | 5-Cl | 6.55 |
| 118 | 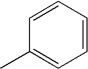 | 5-CH₃O | 2.51 |
| 119 | 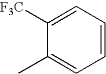 | 5-CH₃O | 2.81 |
| 120 | 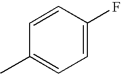 | 5-CH₃O | 2.55 |
| 121 | 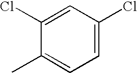 | 5-CH₃O | 3.04 |
| 122 | 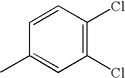 | 5-CH₃O | 3.14 |
| 123 | 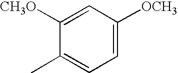 | 5-CH₃O | 2.71 |
| 124 | 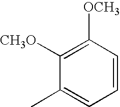 | 5-CH₃O | 2.76 |
| 125 | 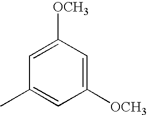 | 5-CH₃O | 2.52 |
| 126 | 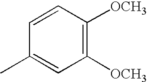 | 5-CH₃O | 2.54 |
| 127 | 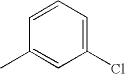 | 5-CH₃O | 2.83 |
| 128 | 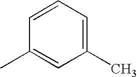 | 5-CH₃O | 2.73 |
| 129 | 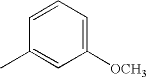 | 5-CH₃O | 2.68 |
| 130 | 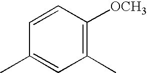 | 5-CH₃O | 2.61 |
| 131 | 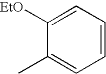 | 5-CH₃O | 2.85 |
| 132 | 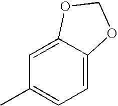 | 5-CH₃O | 2.65 |
| 133 | 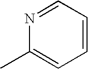 | 5-CH₃O | 1.62 |
| 134 | 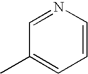 | 5-CH₃O | 0.45 |
| 135 | 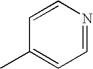 | 5-CH₃O | 1.03 |
| 136 | 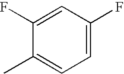 | 5-CH₃O | 3.46 |
| 137 | 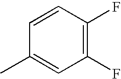 | 5-CH₃O | 3.58 |
| 138 | 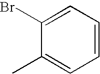 | 5-CH₃O | 3.65 |
| 139 | 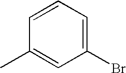 | 5-CH₃O | 3.69 |
| 140 | 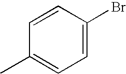 | 5-CH₃O | 3.79 |
| 141 | 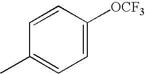 | 5-CH₃O | 3.88 |
| 142 | 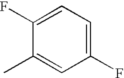 | 5-CH₃O | 3.42 |
| 143 | 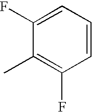 | 5-CH₃O | 3.34 |
| 144 | 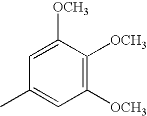 | 5-CH₃O | 3.32 |
| 145 | 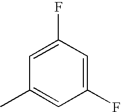 | 5-CH₃O | 3.52 |
| 146 | 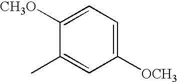 | 5-CH₃O | 3.51 |
| 147 | 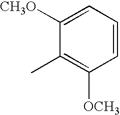 | 5-CH₃O | 3.61 |
| 148 | 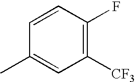 | 5-CH₃O | 3.88 |
| 149 | 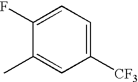 | 5-CH₃O | 3.74 |
| 150 | 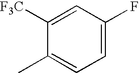 | 5-CH₃O | 3.65 |
| 151 | 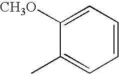 | 5-CH₃O | 5.56 |

The used aralkylamine derivatives are commercial products.

### EXAMPLE 2

### Preparation of Pharmaceutical Compositions

### a) Tablets:

0.01-50% of active ingredient of formula I, 15-50% of lactose, 15-50% of potato starch, 5-15% of polyvinyl pyrrolidone, 1-5% of talc, 0.01-3% of magnesium stearate, 1-3% of colloid silicon dioxide and 2-7% of ultraamylopectin are mixed, then are granulated by wet granulation and pressed to tablets.

b) Dragées, Filmcoated Tablets:

The tablets made according to the method described above are coated by a layer consisting of entero- or gastrosolvent film, or of sugar and talc. The dragées are polished by a mixture of beeswax and carnuba wax.

### c) Capsules:

0.01-50% of active ingredient of formula I, 1-5% of sodium lauryl sulfate, 15-50% of starch, 15-50% of lactose, 1-3% of colloid silicon dioxide and 0.01-3% of magnesium stearate are thoroughly mixed, the mixture is passed through a sieve and filled in hard gelatin capsules.

### d) Suspensions:

Ingredients: 0.01-15% of active ingredient of formula I, 0.1-2% of sodium hydroxide, 0.1-3% of citric acid, 0.05-0.2% of nipagin (sodium methyl 4-hydroxybenzoate), 0.005-0.02% of nipasol, 0.01-0.5% of carbopol (polyacrilic acid), 0.1-5% of 96% ethanol, 0.1-1% of flavoring agent, 20-70% of sorbitol (70% aqueous solution) and 30-50% of distilled water.

To solution of nipagin and citric acid in 20 ml of distilled water, carbopol is added in small portions under vigorous stirring, and the solution is left to stand for 10-12 h. Then the sodium hydroxide in 1 ml of distilled water, the aqueous solution of sorbitol and finally the ethanolic raspberry flavor are added with stirring. To this carrier the active ingredient is added in small portions and suspended with an immersing homogenizator. Finally the suspension is filled up to the desired final volume with distilled water and the suspension syrup is passed through a colloid milling equipment.

### e) Suppositories:

For each suppository 0.01-15% of active ingredient of formula I and 1-20% of lactose are thoroughly mixed, then 50-95% of adeps pro suppository (for example Witepsol 4) is melted, cooled to 35° C. and the mixture of active ingredient and lactose is mixed in it with homogenizator. The obtained mixture is mould in cooled forms.

### f) Lyophilized Powder Ampoule Compositions:

A 5% solution of mannitol or lactose is made with bidistilled water for injection use, and the solution is filtered so as to have sterile solution. A 0.01-5% solution of the active ingredient of formula I is also made with bidistilled water for injection use, and this solution is filtered so as to have sterile solution. These two solutions are mixed under aseptic conditions, filled in 1 ml portions into ampoules, the content of the ampoules is lyophilized, and the ampoules are sealed under nitrogen. The contents of the ampoules are dissolved in sterile water or 0.9% (physiological) sterile aqueous sodium chloride solution before administration.

What we claim is:

1. New indole-2-carboxamidine derivatives of formula (I) 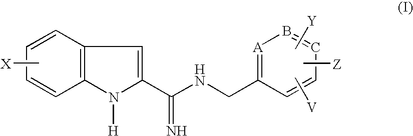
wherein the meaning of
X is hydrogen or halogen atom, C₁-C₄ alkyl, C₁-C₄ alkoxy, trifluoromethyl group,
Y, V and Z independently are hydrogen or halogen atom, hydroxy, cyano, C₁-C₄ alkylsulfonamido optionally substituted by a halogen atom or halogen atoms, C₁-C₄ alkanoylamido optionally substituted by a halogen atom or halogen atoms, trifluoromethyl, trifluoromethoxy, C₁-C₄ alkyl, C₁-C₄ alkoxy group, or
the neighboring V and Z groups in given case together with one or more identical or different additional hetero atom and —CH═ and/or —CH₂— groups can form all optionally substituted 4-7 membered homo- or heterocyclic ring, preferably benzene, dioxolane ring,
A, B and C independently are —CH═ group or one of them is nitrogen atom, and the salts thereof.

2. A compound of the following group of indole-2-carboxamidine derivatives belonging to the scope of claim 1
N-(2,6-dimethoxy-benzyl)-1H-indole-2-carboxamidin,
N-(2-ethoxy-benzyl)-1H-indole-2-carboxamidine,
N-(3-methoxy-benzyl)-5-methoxy-1H-indole-2-carboxamidine,
N-(2,6-dimethoxy-benzyl)-5-chloro-1H-indole-2-carboxamidine,
N-(2,6-dimethoxy-benzyl)-5-methoxy-1H-indole-2-carboxamidine,
N-(2-ethoxy-benzyl)-5-fluoro-1H-indole-2-carboxamidine,
N-(2,6-dimethoxy-benzyl)-5-fluoro-1H-indole-2-carboxamidine
and the salts thereof.

3. Pharmaceutical compositions containing an effective amount of the indole-2-carboxamidine derivatives of formula (I)—wherein the meaning of A, B, C, X, Y, V, Z are as given in claim 1—or the salts thereof as active ingredient and auxiliary materials, which are commonly used in practice, such as carriers, excipients, diluents, stabilizers, wetting or emulsifying agents, pH- and osmotic pressure-influencing, flavoring or aromatizing, as well as formulation-promoting or formulation-providing additives.

4. Process for preparing the indole-2-carboxamidine derivatives of formula (I),—wherein the meaning of A, B, C, X, Y, V, Z are as given in claim —, characterized by
reacting a carboximidic acid alkyl ester salt of formula (II) 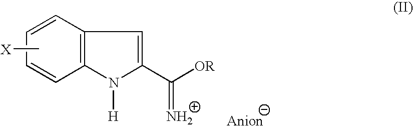
wherein the meaning of X is as given in claim 1, Anion means one equivalent of an anion, R is C₁-C₄ alkyl—with an aralkylamine of formula (III) 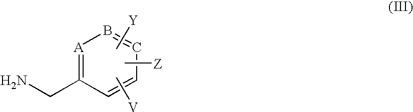
wherein the meaning of A, B, C, Y, V, Z are as given in claim 1—,
then transforming optionally the so obtained indole-2-carboxamidine derivatives of formula (I)—wherein the meaning of A, B, C, X, Y, V, Z are as given in claim 1—into another compounds of formula (I) by introducing new substituents and/or modifying or removing the existing ones, and/or by forming salt and/or liberating the compound of formula (I) from salts by known methods.

5. Process as claimed in claim 4, characterized by reacting the carboximidic acid alkyl ester hydrochloride of formula (II)—wherein the meaning of X and R is as given in claim 1, and Anion means Cl⁻ ion—with the aralkylamine of formula (III)—wherein the meaning of A, B, C, Y, V, Z are as given in claim 1—in the presence of a base.

6. Process as claimed in claim 4, characterized by reacting the carboximidic acid alkyl ester hydrochloride of formula (II)—wherein the meaning of X and R is as given in claim 1, and Anion means Cl⁻ ion—with the aralkylamine of formula (III)—wherein the meaning of A, B, C, Y, V, Z are as given in claim 1—in the presence of excess of the aralkylamine of formula (III)—wherein the meaning of A, B, C, Y, V, Z are as given in claim 1—, in an alkanol.

7. Process for manufacturing pharmaceutical compositions having NR2B selective NMDA receptor antagonist effect, characterized by mixing a indole-2-carboxamidine derivative of formula (I)—wherein the meaning of A, B, C, X, Y, V, Z are as given in claim 1—or the pharmaceutically acceptable salts thereof as active ingredient and auxiliary materials, which are commonly used in practice, such as carriers, excipients, diluents, stabilizers, wetting or emulsifying agents, pH- and osmotic pressure-influencing, flavoring or aromatizing, as well as formulation-promoting or formulation-providing additives.

8. Method of treatment and alleviation of symptoms of the following diseases of mammals—including human—traumatic injury of brain or spinal cord, human immunodeficiency virus (HIV) related neuronal injury, amyotrophic lateral sclerosis, tolerance and/or dependence to opioid treatment of pain, withdrawal syndromes of e.g. alcohol, opioids or cocaine, ischemic CNS disorders, chronic neurodegenerative disorders, such as Alzheimer's disease, Parkinson's disease, Huntington's disease, pain and chronic pain states, such as neuropathic pain or cancer related pain, epilepsy, anxiety, depression, migraine, psychosis, muscular spasm, dementia of various origin, hypoglycemia, degenerative disorders of the retina, glaucoma, asthma, tinnitus, aminoglycoside antibiotic—induced hearing loss, characterized by administering effective amount/amounts of an indole-2-carboxamidine derivative of formula (I)—wherein the meaning of A, B, C, X, Y, V, Z are as given in claim 1—or optical antipodes or racemates or the pharmaceutically acceptable salts thereof as such or combined with carriers, filling materials and the like usually applied in pharmaceuticals to the mammal to be treated.

9. Use of an indole-2-carboxamidine derivative of formula (I)—wherein the meaning of A, B, C, X, Y, V, Z are as given in claim 1—or optical antipodes or racemates or the pharmaceutically acceptable salts thereof for the preparation of a pharmaceutical for the treatment and alleviation of symptoms' of the following diseases in a mammals, including humans: traumatic injury of brain or spinal cord, human immunodeficiency virus (HIV) related neuronal injury, amyotrophic lateral sclerosis, tolerance and/or dependence to opioid treatment of pain, withdrawal syndromes of e.g. alcohol, opioids or cocaine, ischemic CNS disorders, chronic neurodegenerative disorders, such as Alzheimer's disease, Parkinson's disease, Huntington's disease, pain and chronic pain states, such as neuropathic pain or cancer related pain, epilepsy, anxiety, depression, migraine, psychosis, muscular spasm, dementia of various origin, hypoglycemia, degenerative disorders of the retina, glaucoma, asthma, tinnitus, aminoglycoside antibiotic-induced hearing loss.
